(19) United States
(12) Patent Application Publication
(10) Pub. No.: US 2022/0370321 A1
(43) Pub. Date: Nov. 24, 2022

(54) FLAVOR COMPOSITIONS FOR BEVERAGE AND PERSONAL CARE APPLICATIONS

(71) Applicant: Fimenich SA, Satigny (CH)

(72) Inventor: Qiu-Min MA, Shanghai (CN)

(73) Assignee: Firmenich SA, Satigny (CH)

(21) Appl. No.: 17/761,242

(22) PCT Filed: Dec. 14, 2020
(86) PCT No.: PCT/EP2020/085904

§ 371 Date: Mar. 17, 2022

(30) Foreign Application Priority Data
Dec. 16, 2019 (CN) PCT/CN2019/125479
Feb. 4, 2020 (EP) 20155333.6

(51) Int. Cl. A61K 8/58; A23D 7/005; A23D 7/01; A23L 27/00; A23L 27/12; A23L 2/56; A61K 8/34; A61K 8/60; A61Q 11/00; A61K 8/06

(52) U.S. Cl. CPC A61K 8/58; A23D 7/0053; A23D 7/011; A23L 27/80; A23L 27/13; A23L 2/56; A61K 8/345; A61K 8/60; A61Q 11/00; A61K 8/062; A23V 2002/00

(57) ABSTRACT

The present invention relates to a composition comprising a surfactant system comprising lysolecithin and lecithin, or lysolecithin and sugar ester, a non-polar phase comprising a flavor oil, and a polar phase comprising at least one polar non-aqueous solvent, wherein the flavor oil is present in an amount of at least 10 wt. %, based on the total amount of the composition, and wherein lysolecithin is present in an amount of at least 10 wt. %, based on the total amount of the composition. The invention further relates to a method for the preparation of the inventive composition, as well as to the use of the inventive composition for the preparation of a flavored beverage or personal care product. Moreover, the invention concerns a beverage or personal care product comprising the inventive composition.

### TECHNICAL FIELD

The present invention relates to a composition comprising a surfactant system comprising lysolecithin and lecithin, or lysolecithin and sugar ester, a non-polar phase comprising a flavor oil, and a polar phase comprising at least one polar non-aqueous solvent, wherein the flavor oil is present in an amount of at least 10 wt. %, based on the total amount of the composition, and wherein lysolecithin is present in an amount of at least 10 wt. %, based on the total amount of the composition. The invention further relates to a method for the preparation of the inventive composition, as well as to the use of the inventive composition for the preparation of a flavored beverage or personal care product. Moreover, the invention concerns a beverage or personal care product comprising the inventive composition.

### BACKGROUND OF THE INVENTION

For beverage and personal care applications, flavor compositions are desired.

Flavor compositions can be in the form of emulsions comprising an aqueous phase, an oil phase, as well as a surfactant system. The aqueous phase commonly comprises water and optionally one or more polar co-solvents and further ingredients. The oil phase is commonly dispersed within the aqueous phase thereby forming an oil-in-water emulsion. The dispersed oil phase typically comprises flavor oil(s) and optionally further lipophilic ingredients.

WO 2011/089249 A1 discloses a composition comprising an aqueous phase, a surfactant system and an oil phase. The surfactant system comprises one or more saponins, and lecithin.

WO 2016/064828 Al discloses a metastable, translucent flavor nanoemulsion that contains a flavor oil phase, an aqueous phase, and a surfactant system having a first lecithin and a second lecithin.

US 2017/0311632 A1 discloses a flavor emulsion prepared from a lysolecithin composition, and a liquid beverage or liquid beverage concentrate containing the lysolecithin composition.

US 2015/0030748 A1 discloses a method of making a clear beverage concentrate, which contains a nanoemulsion of a liquid flavor oil in an aqueous phase, including the emulsification of the flavor oil into water in the presence of an emulsifier, which emulsifier consists of at least 5% by weight quillaja saponins.

US 2009/0285952 A1 discloses a transparent emulsified composition and transparent emulsified flavor composition containing enzyme-degraded lecithin, for use in alcoholic drinks or carbonated drinks.

The present invention is able to provide a flavor composition, which is clear in appearance after dilution, but which is also stable, both in concentrated and in diluted form (within a beverage or personal care product). The stability is ensured over a wide range of temperatures and storage conditions. Nanoemulsions of the prior art show little thermodynamic stability and high-energy input is required for their production.

Moreover, the present invention is able to provide a flavor composition with a high oil and low water proportion. A high oil proportion is desirable as the flavor composition is able to effectively and efficiently provide flavor to a beverage, food or consumer product. A low water content is desirable as flavor compounds are chemically more stable at low water contents, or even in water-free systems. However, the production of a stable emulsion showing a high oil content is challenging, in particular in view of its stability during storage. Moreover, in the case of nanoemulsions, high oil proportions lead to an increased viscosity, which complicates further processing. Therefore, known emulsions are usually restricted to a low oil content to ensure sufficient stability.

Moreover, the present invention is able to provide a flavor composition that shows good stability at acidic pH levels. As the pH-value of many beverages is commonly at acidic levels, good stability of the flavor emulsions under acidic conditions is advantageous. However, many emulsions according to the prior art are hardly stable under acidic conditions, as e.g. the surfactant used is not stable under acidic conditions.

The present invention provides solutions in order to provide the above-mentioned advantages and to overcome the above-mentioned disadvantages associated with the known flavor compositions.

### DETAILED DESCRIPTION OF THE INVENTION

The present invention relates to a composition comprising

a surfactant system comprising lysolecithin and lecithin, or lysolecithin and sugar ester,

a non-polar phase comprising a flavor oil,

a polar phase comprising at least one polar non-aqueous solvent; wherein the flavor oil is present in an amount of at least 10 wt. %, based on the total amount of the composition, and wherein lysolecithin is present in an amount of at least 10 wt. %, based on the total amount of the composition.

In a particular embodiment, the composition is an emulsion. An emulsion is to be understood as a mixture of two lipids that are immiscible due to their different polarities (hydrophobic vs. hydrophilic). In an emulsion, one liquid (dispersed or internal phase) is dispersed in another liquid (external or continuous phase). Therefore, the non-polar phase may be dispersed within the polar-phase, or the polar phase may be dispersed within the non-polar phase.

In a particular embodiment, the non-polar phase is dispersed within the polar phase and, in another particular embodiment, the polar phase is dispersed within the non-polar phase. Preferably, the non-polar phase is dispersed within the polar phase.

In a particular embodiment, the emulsion is a microemulsion. Microemulsions including micellar solutions are usually transparent dispersions that form spontaneously without the need of energy input, when the compounds thereof are properly mixed with each other. Due to the very small size of dispersed oil-droplets, the dispersed oil-droplets are less than 140 nm in diameter, the visible light cannot be scattered and therefore microemulsions appear as clear or translucent isotropic solutions. A classical oil-in-water microemulsion consists of water, a co-solvent such as alcohol, oil and one or more surfactants and co-surfactants. A high proportion of oil is feasible for microemulsion systems, which saves transportation and storage costs.

By contrast, nanoemulsions are usually prepared by high-energy input, such as high-pressure homogenization to break the big droplets into small ones. The size of the oil-droplets is usually below 500 nm, or even 200 nm for clear beverage applications. Nanoemulsions are not thermodynamically stable, which means that they separate into their two original liquid phases on standing mainly through Ostwald ripening. In addition, the oil proportion of nanoemulsions is low, because high oil proportions would increase the viscosity of the system and make it difficult to process. The high-energy input and the low oil proportions increase the costs of flavor nanoemulsion products. The composition of the present invention is preferably not in the form of a nanoemulsion.

The composition according to the present invention comprises a surfactant system comprising lysolecithin and lecithin, or lysolecithin and sugar ester.

A surfactant system is required to obtain a composition that is at least thermodynamically stable for a certain period of time (e.g. for one month). Surfactants (emulsifiers) show amphiphilic properties meaning that they contain both hydrophobic and hydrophilic moieties. Based on these structural properties, surfactants are surface-active, which allows them to reduce the interfacial tension between a polar and non-polar phase and thus, to stabilize an emulsion.

According to the present invention, the surfactant system comprises lysolecithin and lecithin, or lysolecithin and sugar ester as surfactants.

The lysolecithin to be used in the present invention is not particularly restricted. For example, use can be made of enzyme-modified lecithin, enzyme-treated lecithin or enzyme-hydrolyzed lecithin which contains as the main component lysolecithin (1-monoacylglycerophospholipid), which is obtained by enzymatically hydrolyzing natural substance-derived lecithin (1,2-diacylglycerophospholipid) including, but not limited to lecithin from soybeans, eggs, sunflower or rapeseed (canola) seeds, milk, marine sources, and cottonseeds, and then eliminating the thus formed free fatty acids and fat-soluble components originating in the starting materials. The enzyme to be used herein is not particularly restricted and exemplified by phospholipase, lipase, etc. The lysolecithin according to the invention is soluble in polar solvents, such as polar non-aqueous solvents or water.

Preferably, lysolecithin is prepared by hydrolyzing a lecithin under a hydrolysis process with phospholipase A1 or A2.

Lecithins preferably used for the preparation of lysolecithin are those containing a high content of phospholipids. Preferably the proportion of phosphatidylcholines in the lecithin is at least 50 wt. %, based on the total amount of phospholipids.

In a particular embodiment, the lysolecithin content in the enzymatically treated lecithin mixture is higher than 50 wt. %, preferably higher than 80 wt. %, based on the total amount of phospholipids.

Preferably, the lysolecithin of the present invention is selected from the group consisting of lysophosphatidylcholine, lysophosphatidylethanolamine, lysophosphatidylinositol, lysophosphatidylgylcerol and lysophosphatidylserine or a mixture of two or more thereof. Preferably, the lysolecithin of the present invention contains 50% by weight or more, more preferably, contains 80% by weight or more of the lysolecithin component based on the total phospholipids.

According to the invention, lysolecithin is present in the composition in an amount of at least 10 wt. %, based on the total amount of the composition.

In a particular embodiment, lysolecithin is present in the composition in an amount of at least 20 wt. %, based on the total amount of the composition.

In a particular embodiment, lysolecithin is present in the composition in an amount of at least 40 wt. %, based on the total amount of the composition.

In a particular embodiment, lysolecithin is present in the composition in an amount of from 10 to 60 wt. %, based on the total amount of the composition.

In a particular embodiment, lysolecithin is present in the composition in an amount of from 10 to 40 wt. %, based on the total amount of the composition.

In a particular embodiment, lysolecithin is present in the composition in an amount of from 10 to 30 wt. %, preferably from 15 to 25 wt. %, based on the total amount of the composition.

In one alternative of the invention, the surfactant system further comprises lecithin next to lysolecithin.

Lecithins are mixtures of two primary components, namely phospholipids and triglycerides, with minor amounts of other constituents such as phytoglycolipids, phytosterols, tocopherols, and fatty acids. Phospholipids in lecithin include positively charged phospholipids (e.g. phosphatidylcholine, phosphatidylethanolamine, and phosphatidylserine) and negatively charged phospholipids (e.g. phosphatidic acid, phosphatidylglycerol, and phosphatidylinositol). Lecithins are prepared by extracting and purifying phospholipids from naturally occurring products including, but not limited to, soybeans, eggs, sunflower or rapeseed (canola) seeds, milk, marine sources, and cottonseeds. Lecithins may be deoiled (i.e., having 3% or less residual oil), or fractionated (i.e., separating soluble components and insoluble components in a solvent, which can be an alcohol such as ethanol or an ethanol-water mixture). Food-grade lecithin are available in liquid, granular and powder from commercial sources. The term “lecithin” as used herein refers to both a single type of lecithin (e.g., native, deoiled, fractionated) as well as to a mixture of lecithins.

In a particular embodiment, the mass ratio of lysolecithin and lecithin is between 20:0 to 1:5, preferably between 10:1 to 1:2.

In a particular embodiment, lecithin is present in the composition in an amount of between 10 and 40 wt. %, based on the total amount of the composition, preferably between 20 and 25 wt. %.

In another alternative of the invention, the surfactant system further comprises a sugar ester next to lysolecithin.

The sugar ester acts as another surfactant next to lysolecithin. Preferably, the sugar ester is a sucrose ester of fatty acids, or a mixture of sucrose esters of fatty acids. Preferably, the fatty acids are selected from the group consisting of palmitic acid, myristic acid, lauric acid, oleic acid and stearic acid.

In a particular embodiment, the mass ratio of lysolecithin and the sugar ester is between 20:0 to 1:5, preferably between 10:1 to 1:2.

In a particular embodiment, sugar ester is present in the composition in an amount of between 1 and 10 wt. %, based on the total amount of the composition, preferably between 3 and 7 wt. %.

The composition according to the present invention further comprises a non-polar phase comprising a flavor oil.

Under “non-polar phase” is to be understood the total amount of hydrophobic compounds in the composition according to the invention.

In a particular embodiment, the non-polar phase may further include one or more other active ingredients selected from oil-soluble pharmaceutical ingredients, oil-soluble nutraceutical ingredients (e.g., oil-soluble vitamins), oil-soluble colorants, oil-soluble antimicrobial ingredients, oil-soluble defoamers, mouthfeel modulators, taste modulators, and any combinations thereof. Useful taste modulators include acid maskers, polyaldo matric, beer hops, cooling agents, hot tasting substances, sweet enhancers, salt enhancers, salivation-inducing substances, substances causing a warmth or tingling feeling, and any combinations thereof. Exemplary mouthfeel modulators are coconut oil, coconut milk with or without sugar, vanillin, stevia glycosides such as Rebaudiosides A, C, D, E, and F, medium chain triglycerides, steviol, glucosylated stevia glycosides, and combinations thereof. At least one cooling compound is selected from the group consisting of: 2-(4-ethylphenoxy)-N-(1H-pyrazol-5-yl)-N-(2-thienylmethyl)acetamide, WS-23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (1R,2S,5R)-N-(4-Methoxyphenyl)-p-menthanecarboxamide, FEMA 4681; WS-27 (N-Ethyl-2,2-diisopropylbutanamide), FEMA 4557; N-Cyclopropyl-5-methyl-2-isopropylcyclohexanecarboxamide, FEMA 4693, WS-116 (N-(1,1-Dimethyl-2-hydroxyethyl)-2,2-diethylbutanamide), N-(1, 1-Dimethyl-2-hydroxyethyl)2,2-diethylbutanamide, FEMA 4603, Menthoxyethanol, FEMA 4154, N-(4-cyanomethylphenyl)-p-menthanecarboxamide, FEMA 4496; N-(2-(Pyridin-2-yl)ethyl)-3-p-menthanecarboxamide, FEMA 4549; N-(2-Hydroxyethyl)-2-isopropyl-2,3-dimethylbutanamide, FEMA 4602 and (also N-(4-(carbamoylmethyl)phenyl)-menthylcarboxamide, FEMA 4684; (1R,2S,5R)-N-(4-Methoxyphenyl)-p-menthanecarboxamide (WS-12), FEMA 4681; (2S,5R)-N-[4-(2-Amino-2-oxoethyl)phenyl]-p-menthanecarboxamide, FEMA 4684; and N-Cyclopropyl-5-methyl-2-isopropylcyclohexanecarbonecarboxamide, FEMA 4693; 2-[(2-p-Menthoxy)ethoxy]ethanol, FEMA 4718; (2,6-Diethyl-5-isopropyl-2-methyltetrahydropyran, FEMA 4680);trans-4-tert-Butylcyclohexanol, FEMA 4724; 2-(p-tolyloxy)-N-(1H-pyrazol-5-yl)-N-((thiophen-2-yl)methyl)acetamide, FEMA 4809; Menthone glycerol ketal, FEMA 3807; Menthone glycerol ketal, FEMA 3748; (−)-Menthoxypropane-1,2-diol; 3-(l-Menthoxy)-2-methylpropane-1,2-diol, FEMA 3849; Isopulegol; (+)-cis & (−)-trans p-Menthane-3,8-diol, Ratio ˜62:38, FEMA 4053; 2,3-dihydroxy-p-menthane; 3,3,5-trimethylcyclohexanone glycerol ketal; menthyl pyrrolidone carboxylate; (1R,3R,4S)-3-menthyl-3,6-dioxaheptanoate; (1R,2S,5R)-3-menthyl methoxyacetate; (1R,2S,5R)-3-menthyl 3,6,9-trioxadecanoate; (1R,2S,5R)-3-menthyl 3.6,9-trioxadecanoate; (1R,2S,5R)-3-menthyl (2-hydroxyethoxy)acetate; (1R,2S,5R)-menthyl 11-hydroxy-3,6,9-trioxaundecanoate; Cubebol, FEMA 4497; N-(4-cyanomethylphenyl) p-menthanecarboxamide, FEMA 4496; 2-isopropyl-5-methylcyclohexyl 4-(dimethylamino)-4-oxobutanoate, FEMA 4230; N-(4-cyanomethylphenyl) p-menthanecarboxamide, FEMA 4496; N-(2-pyridin-2-ylethyl) p-;menthanecarboxamide, FEMA 4549, Menthyl lactate, FEMA 3748; 6-isopropyl-3,9-dimethyl-1,4-dioxaspiro[4.5]decan-2-one, FEMA 4285; N-benzo[1,3] dioxol-5-yl-3-p-menthanecarboxamide; N-(1-isopropyl-1,2-dimethylpropyl)-1,3-benzodioxole-5-carboxamide; N-(R)-2-oxotetrahydrofuran-3-yl-(1R,2S,5R)-p-menthane-3-carboxamide; mixture of 2,2,5,6,6-pentamethyl-2,3,6,6a-tetrahydropentalen-3a(1H)-ol and 5-(2-hydroxy-2-methylpropyl)-3,4,4-trimethylcyclopent-2-en-1-one; (1R,2S,5R)-2-isopropyl-5-methyl-N-(2-(pyridin-2-yl)ethyl)cyclohexanecarboxamide, FEMA 4549; (2S,5R)-2-isopropyl-5-methyl-N-(2-(pyridin-4-yl)ethyl)cyclohexanecarboxamide; N-(4-cyanomethylphenyl) p-menthanecarboxamide, FEMA 4496; (1S,2S,5R)-N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-yl)ethyl]benzenesulfonamide; 4-cyano-N-phenyl-N-[2-(pyridin-2-yl)ethyl]benzenesulfonamid; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol, 4((9H-fluoren-9-ylamino)methyl)-2-methoxyphenol; 4-((benzhydrylamino)methyl)-2-ethoxyphenol; 1-(4-methoxyphenyl)-2-(1-methyl-1H-benzo[d]imidazol-2-yl)vinyl4-methoxybenzoate; 2-(1-isopropyl-6-methyl-1H-benzo[d]imidazol-2-yl)-1-(4-methoxyphenyl)vinyl4-methoxybenzoate; (Z)-2-(1-isoprop yl-5-methyl-1H-benzo[d]imidazol-2-yl)-1-(4-methoxy-phenyl)vinyl-4-methoxybenzoate; 3-alkyl-p-methan-3-ol derivatives; derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl; menthyl oxamate derivatives; menthyl 3-oxocarboxylic acid esters; N alpha-(Menthanecarbonyl)amino acid amides; p-menthane carboxamide and WS-23 analogs; (−)-(1R,2R,4S)-dihydroumbellulol; p-menthane alkyloxy amides; cyclohexane derivatives; butone derivatives; a mixture of 3-menthoxy-1-propanol and 1-menthoxy-2-propanol; 1-[2-hydroxyphenyl]-4-[2-nitrophenyl]-1,2,3,6-tetrahydropyrimidine-2-one; 4-methyl-3-(1-pyrrolidinyl)-2[5H]-furanone; and combinations thereof. In a particular embodiment, the non-polar phase is present in the composition in an amount of from 20 to 50 wt. %, more preferably from 30 to 46 wt. %, based on the total amount of the composition.

By “flavor oil”, it is meant here a flavouring ingredient or a mixture of flavouring ingredients, solvent or adjuvants of current use for the preparation of a flavouring formulation, i.e. a particular mixture of ingredients which is intended to be added to a composition to impart, improve or modify its organoleptic properties, in particular its flavour and/or taste. Taste modulator as also encompassed in said definition. Flavouring ingredients are well known to a skilled person in the art and their nature does not warrant a detailed description here, which in any case would not be exhaustive, the skilled flavourist being able to select them on the basis of his general knowledge and according to the intended use or application and the organoleptic effect it is desired to achieve. Many of these flavouring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor

Ingredients, 1975, CRC Press or Synthetic Food Adjuncts, 1947, by M. B. Jacobs, can Nostrand Co., Inc. Solvents and adjuvants or current use for the preparation of a flavouring formulation are also well known in the art.

In a particular embodiment, the flavor oil comprises thymol, [(CH3)2CHC6H3(CH3)OH, also known as isopropyl-m-cresol], Methyl salicylate [C6H4OHCOOCH3, also known as wintergreen oil], Eucalyptol (C10H18O, also known as cineol), and Menthol (CH3C6H9(C3H7)OH, also known as hexahydrothymol).

In a particular embodiment, the flavor oil is present in the non-polar phase in an amount of at least 40 wt. %, preferably from 50 to 100 wt. %, more preferably from 70 to 100 wt. %, based on the total amount of non-polar phase.

In a particular embodiment, the flavor oil is present in the composition in an amount of at least 20%, preferably from 20 to 50 wt. %, more preferably from 30 to 46 wt. %, based on the total amount of the composition.

The composition according to the present invention further comprises a polar phase comprising at least one polar non-aqueous solvent.

Under “polar phase” is to be understood the total amount of hydrophilic compounds in the composition according to the invention.

In a particular embodiment, the polar phase is present in the composition in an amount of at least 10 wt. %, preferably from 10 to 60 wt. %, more preferably from 20 to 40 wt. %, based on the total amount of the composition.

According to the invention, the polar phase comprises at least one polar non-aqueous solvent.

Under “polar non-aqueous solvent” is to be understood a polar (hydrophilic) solvent that is not water. According to a preferred embodiment, the non-aqueous solvent is a food grade solvent, in particular a non-aqueous solvent for the use for food compositions, in particular in combination with flavor ingredients.

In a particular embodiment, the polar non-aqueous solvent is selected from the group consisting of glycerol, propylene glycol, benzylic alcohol, ethanol, propanol, isopropanol, 1,3-propanediol, butanol, butylene glycol, hexylene glycol, dipropylene glycol, ethoxydiglycol, triacetine, triethylcitrate, C₁₋₆ linear/branched alkyl lactate (e.g. ethyl lactate), xylitol, sorbitol, mannitol, maltitol, inositol, allitol, altritol, dulcitol, galactitol, glucitol, hexitol, iditol, pentitol, ribitol, erythritol, or any mixtures thereof. Preferably, the polar non-aqueous solvent is selected from the group consisting of glycerol, propylene glycol, ethyl lactate, ethanol or sorbitol, more preferably the non-aqueous solvent is propylene glycol.

In a particular embodiment, the polar non-aqueous solvent is present in the polar phase in an amount of at least 50 wt. %, preferably from 75 to 100 wt. %, more preferably from 80 to 100 wt. %, based on the total amount of the polar phase. By definition, the potentially remaining percentage proportions refer to the water content in wt. % based on the total amount of the polar phase.

In a particular embodiment, the polar phase of the composition comprises water. Preferably water is present in the polar phase in an amount of less than 50 wt. %, preferably less than 25 wt. %.

In a particular embodiment, the polar non-aqueous solvent is present in the composition in an amount of from 10 to 60 wt. %, preferably from 20 to 40 wt. %, based on the total amount of the composition.

In a particular embodiment, water is present in the composition in an amount of less than 50 wt. %, preferably less than 20 wt. %, based on the total amount of the composition.

In a particular embodiment of the invention, the composition further comprises one or more antioxidants. Generally, antioxidants are chemical compounds that inhibit oxidation. Antioxidants can be classified into two groups, depending on whether they are soluble in polar solvents (hydrophilic antioxidants) or in non-polar solvents (lipophilic antioxidants). Examples for hydrophilic antioxidants are ascorbic acid (Vitamin C), green tea extract, water soluble rosemary extract, glutathione, lipoic acid, and uric acid. Examples for hydrophobic antioxidants are carotenes, tocopherols (Vitamin E), butylated hydroxyanisole (BHA), butylated hydroytoluene (BHT), oil soluble rosemary extract and ubiquinol (coenzyme Q). In case the composition is an emulsion, the antioxidant will either be present in the polar or non-polar phase depending on its hydrophilic/lipophilic properties.

In a particular embodiment of the invention, the composition further comprises one or more vitamins. A vitamin is an organic molecule (or related set of molecules) that is an essential micronutrient that an organism needs in small quantities for the proper functioning of its metabolism. Similar to what has been said above for antioxidants, vitamins can be classified into two groups, depending on whether they are soluble in polar solvents (hydrophilic vitamins) or in non-polar solvents (lipophilic vitamins). Examples for hydrophilic vitamins are Vitamin C (ascorbic acid) and the B-Vitamins (e.g. B1, B2, B3, B5, B6, B7, and B12). Examples for hydrophobic vitamins are Vitamin A, Vitamin D, Vitamin E, and Vitamin K. In case the composition is an emulsion, the vitamin will either be present in the polar or non-polar phase depending on its hydrophilic/lipophilic properties. In some cases chemicals compounds are both antioxidants and vitamins, such as Vitamin C (ascorbic acid), Vitamin E (tocopherols).

In a particular embodiment of the invention, the composition further comprises one or more sugars. Sugar is the generic name for sweet-tasting carbohydrates that are soluble in polar solvents, such as water. In particular, the term “sugar” refers to monosaccharides, such as glucose, fructose, and galactose, as well as disaccharides, such as sucrose, lactose, and maltose. Longer chains of sugar molecules are not regarded as sugars, and are called oligosaccharides or polysaccharides. Due to their hydrophilic character, sugar compounds are present in the polar phase in case of an emulsion.

In a particular embodiment of the invention, the composition further comprises one or more defoamers. A defoamer or an anti-foaming agent is a chemical additive that reduces and hinders the formation of foam, in particular in liquids used for industrial processes. A defoamer can be a water-dispersible defoamer or oil-soluble defoamer. Examples include silicone emulsion antifoamers, polydimethylsiloxane antifoamers, 2-octanol, petrolatum, hop lipids, alginates, mineral oil, sorbitan monostearate, and a combination thereof.

In a particular embodiment, the composition is clear in appearance, i.e. a clear composition. Compositions having a NTU value of less than 10 are considered to have a clear appearance.

In a particular embodiment, the composition has an instability index of less than 0.1, preferably between 0.01 and 0.07. Preferably, the instability index is determined by using a LumiSizer (LUM GmbH, Germany). Thereby, the instability index is preferably determined at 20 to 25° C. at a centrifugation speed of 4000 rpm, wherein transmission is preferably detected at 870 nm.

The present invention further relates to a method for preparing a composition as described, comprising the steps of:

a) Mixing a non-polar phase comprising a flavor oil and a polar phase comprising at least one polar non-aqueous solvent in the presence of a surfactant system comprising lysolecithin and lecithin, or lysolecithin and sugar ester.

b) Stirring of the mixture obtained after step a).

In a particular embodiment, either step a) or step b) individually, or both step a) and b) take place at a temperature of from 20 to 25° C.

In a particular embodiment, the method for preparing a composition according to the invention does not comprise a high-pressure homogenization step. High-pressure homogenization requires high energy-input, which is to be avoided. Moreover, in case of an emulsion, high-pressure homogenization may lead to the formation of a nanoemulsion that is thermodynamically not stable.

The present invention further relates to the use of a composition according to the invention for the preparation of a flavored beverage or personal care product.

The composition according to the present invention may be used for the preparation of a flavored beverage.

The term “flavored beverage” includes flavored and cream sodas, powdered soft drinks, as well as liquid concentrates such as fountain syrups and cordials; coffee and coffee-based drinks, coffee substitutes and cereal-based beverages; teas, including dry mix products as well as ready-to-drink teas (herbal and tealeaf based); fruit and vegetable juices and juice flavored beverages as well as juice drinks, nectars, concentrates, punches and “ades”; sweetened and flavored waters, both carbonated and still; sport/energy/health drinks; alcoholic beverages plus alcohol-free and other low-alcohol products including beer and malt beverages, cider, and wines (still, sparkling, fortified wines and wine coolers); other beverages processed with heating (infusions, pasteurization, ultra-high temperature, ohmic heating or commercial aseptic sterilization) and hot-filled packaging; and cold-filled products made through filtration or other preservation techniques.

In case the composition is used for the preparation of a flavored beverage, the compounds being comprised by the composition have to be selected such that they are suitable for human consumption. In particular, the polar non-aqueous solvents have to be selected such that they are suitable for human consumption. Therefore, butylene glycol and hexylene glycol should not be present in an inventive composition that is used for the preparation of a flavored beverage.

The composition according to the present invention may also be used for the preparation of a personal care product.

Personal care products are typically applied to the human body for the purposes of cleaning, beautifying, promoting attractiveness or changing its appearance. Personal care products are for example, toothpaste or mouthwashes.

In a particular embodiment, the inventive composition is used for the preparation of a mouthwash. Mouthwashes or mouth rinses are liquid oral care preparations developed to clean and refresh the oral cavity or oral surface by inhibiting or killing the microorganisms that cause malodor, dental caries, tooth decay, gum diseases, gingivitis, and periodontal disorders.

The present invention also relates to a method for preparing a flavored beverage or personal care product comprising the step of:

adding the composition according to the invention to a beverage or personal care product.

The present invention also relates to a beverage or personal care product comprising a composition according to the invention.

In a particular embodiment, the beverage comprises the inventive composition in amounts of from 0.002 to 5 wt. ‰, preferably from 0.02 to 2.5 wt. ‰, based on the total amount of the beverage.

In a particular embodiment, personal care product comprises the inventive composition in amounts of from 0.002 to 2.5 wt. %, preferably from 0.02 to 1.25 wt. %, based on the total amount of the personal care product.

In a particular embodiment, the personal care product is a mouthwash. The mouthwash can be an alcoholic or alcohol-free mouthwash.

In a particular embodiment, the beverage comprises a flavor oil in amounts of from 0.0001 to 0.1 wt. %, preferably from 0.001 to 0.05 wt. %, based on the total amount of the beverage.

In a particular embodiment, the personal care product comprises a flavor oil in amounts of from 0.001 to 0.5 wt. %, preferably from 0.01 to 0.25 wt. %, based on the total amount of the personal care product.

In a particular embodiment, the beverage or personal care product shows a turbidity (NTU) of less than 10, preferably between 0.1 and 9, more preferably between 0.1 and 5.

The NTU-value refers to “Nephelometric Turbidity Units” that are representative for the turbidity of a composition, and are measured by means of a turbidimeter as specified by the United States Environmental Protection Agency. Preferably, turbidity is measured by a portable turbidity meter (Hanna instruments, Woonsocket, R.I., H193703). Generally, beverages or personal care products having a NTU value above 15 can be considered hazy and not clear. By contrast, beverages and personal care products having a NTU of less than 10 can be considered to have a clear appearance.

In a particular embodiment, the beverage or personal care product shows a droplet size of the dispersed phase of between 10 and 140 nm, preferably between 20 and 100 nm. Droplet sizes are preferably measured by a Zetasizer nano ZS (Malvern Instruments Limited, Worcs, UK).

In a particular embodiment, the beverage is an alcoholic or non-alcoholic beverage, preferably the beverage is a non-alcoholic beverage.

In a particular embodiment, the beverage shows a pH-value of from 2.5 to 7, preferably from 2.6 to 4.0.

In a particular embodiment, the beverage shows a pH-value of from 2.6 to 3.5.

As the inventive composition shows sufficient stability under acidic conditions, it can also be present in a beverage showing acidic pH-values.

In a particular embodiment, the beverage is stable for 1 month at a temperature of from 20 to 25° C.

In view of the above, the present invention provides a composition comprising a polar and non-polar phase as well as a surfactant system that allows for high contents of flavor oil in the composition. High oil contents have not been described in the prior art as this goes along with a couple of challenges, in particular stability and processing issues. However, high oil contents allow the application of more flavor oil in one-step, which lowers application and storage costs. Furthermore, flavor compounds are chemically more stable at high oil contents.

Although the inventive composition comprises high amounts of flavor oil, a final product, such as a beverage or personal care product, can be obtained upon dilution that is clear in appearance and that shows good stability during storage.

Therefore, a particular objective technical problem underlying the present invention is the provision of a composition comprising a polar and non-polar phase, and a surfactant system, that shows a high amount of flavor oil.

A further particular objective technical problem underlying the present invention is the provision of a composition comprising a polar and non-polar phase, and a surfactant system, that shows good stability during storage, also at acidic pH values.

A yet further particular objective technical problem underlying the present invention is the provision of a composition showing a high amount of flavor oil that can be used for the preparation of a clear beverage or personal care product.

A yet further particular objective technical problem underlying the present invention is the provision of a composition showing a high amount of flavor oil that can be used for the preparation of a beverage or personal care product that is stable during subsequent storage.

### EXAMPLES

1. Preparation of flavor microemulsions

Flavor microemulsions according to Samples 1 to 7 were prepared by mixing all the ingredients shown in Table 1 together on stir plates at a temperature between 20 and 25° C.

Flavor microemulsions according to Samples 8 to 14 were prepared by mixing all the ingredients shown in Table 2 together on stir plates at a temperature between 20 and 25° C.

TABLE 1
Flavor microemulsion compositions.

| | Sample | | | | | | |
|---|---|---|---|---|---|---|---|
| | 1 | 2 | 3 | 4 | 5 | 6 | 7 |
| Ingredient | % wt | % wt | % wt | % wt | % wt | % wt | % wt |
| Lime oil | 36 | 45.5 | | | | | |
| Grapefruit oil | | | 36 | 36 | | | |
| Lemon oil | | | | | 36 | | |
| Fresh lime | | | | | | 36 | 36 |
| S LPC80 | 19.2 | 19.6 | 21 | 20.57 | 18 | 18 | 18 |
| LIPOID P 45 | 4.8 | 4.9 | | | 6 | | |
| LIPOID H 100 | | | 3 | 3.43 | | 6 | |
| Emulpur ® SF | | | | | | | 6 |
| Propylene glycol | 40 | 30 | 40 | 40 | 40 | 40 | 40 |
| Total | 100 | 100 | 100 | 100 | 100 | 100 | 100 |

TABLE 2
Flavor microemulsion compositions.

| | Sample | | | | | | |
|---|---|---|---|---|---|---|---|
| | 8 | 9 | 10 | 11 | 12 | 13 | 14 |
| Ingredient | % wt | % wt | % wt | % wt | % wt | % wt | % wt |
| Lime oil | | | | | 35 | 40 | 45.5 |
| Mikan flavor | 42 | 40 | 42 | 35 | | | |
| S LPC80 | 22.4 | 36 | 22.4 | 31.5 | 28 | 36 | 19.6 |
| F160 | 5.6 | | | | 7 | | |
| PS750 | | 4 | 5.6 | | | | |
| F110 | | | | 3.5 | | 4 | |
| F50 | | | | | | | 4.9 |
| Propylene glycol | 30 | 20 | 30 | 30 | 30 | 20 | 30 |
| Total | 100 | 100 | 100 | 100 | 100 | 100 | 100 |

2. Preparation of comparative flavor nanoemulsions

Flavor nanoemulsions using different kinds of lecithin as described in WO2016064848 were prepared as comparative examples. The oil phases were prepared by mixing the flavor with standard liquid lecithin (GIRALEC). The aqueous phases were prepared by adding Lipoid lecithin H50 (comparable to ALCOLEC PC 50 in WO2016064848) and/or standard de-oiled lecithin Emulpur® SF in water solution comprising glycerol, propylene glycol and sorbitol. Pre-emulsions were formed by mixing the oil phases and aqueous phases using high-speed homogenization at 6500 rpm for 3 min. The final nanoemulsions were prepared by homogenizing the pre-emulsions at 7000/725 psi for 3 passes. The compositions are shown in Table 3.

TABLE 3
Comparative flavor nanoemulsion compositions.

Sample

| | Comparative 1 | Comparative 2 |
|---|---|---|
| Ingredient | % wt | % wt |
| Lime oil | 3 | 3 |
| GIRALEC | 0.6 | 0.3 |
| Lipoid Lecithin H50 | 1 | 1 |
| Emulpur ® SF | — | 0.3 |
| Glycerol | 25.4 | 25.4 |
| Propylene glycol | 10 | 10 |
| Sorbitol solution (70%) | 60 | 60 |
| Total | 100 | 100 |

Explanations given for the ingredient abbreviations are summarized in Table 4 below:

Ingredient

| | |
|---|---|
| S LPC80 | Lysolecithin product contains at least 80% wt of |
| | lysolecithin (ex. Lipoid GmbH) |
| LIPOID H 100 | Sunflower lecithin contains not less than 90% wt of |
| | phosphatidylcholine (ex. Lipoid GmbH) |
| LIPOID P 45 | Fat free soybean lecithin with 45% phosphatidylcholine |
| | from non-genetically-modified plants (ex. Lipoid GmbH) |
| Lipoid H 50 | Fat-free sunflower lecithin with 45% phosphatidylcholine |
| | from non-genetically-modified plants (ex. Lipoid GmbH) |
| Emulpur ® SF | De-oiled, fine powdered sunflower lecithin (ex. Cargill) |
| GIRALEC | Standard liquid lecithin (ex. Lasenor) |
| F160 | Sugar ester contains 70% sucrose monostearate |
| | (ex. DK ESTER) |
| PS750 | Sugar ester contains 75% sucrose monopalmitate |
| | (ex. Sisterna) |
| F110 | Sugar ester contains 50% sucrose monostearate |
| | (ex. DK ESTER) |
| F50 | Sugar ester contains 30% sucrose monostearate |
| | (ex. DK ESTER) |

3. Preparation of beverages using the flavor microemulsions according to Samples 1 to 14 and Comparative Samples 1 and 2

Beverages A to N according to the invention were prepared by diluting the flavor microemulsions of Samples 1 to 14, respectively, in a sugar acid beverage base to a final flavor oil concentration of 0.05 wt. %. The same way, comparative beverages O and P were prepared using comparative flavor nanoemulsions 1 and 2. The composition of the beverages according to the invention are given in Tables 5 and 6. The composition of the comparative beverages is given in Table 7. All beverages showed a pH-value of 2.8.

TABLE 5
Beverage compositions A to G.

| | A | B | C | D | E | F | G |
|---|---|---|---|---|---|---|---|
| | Mass | Mass | Mass | Mass | Mass | Mass | Mass |
| Ingredients | (g) | (g) | (g) | (g) | (g) | (g) | (g) |
| Flavor | 1.39 | 1.10 | 1.39 | 1.39 | 1.39 | 1.39 | 1.39 |
| microemulsion | | | | | | | |
| Sugar | 60 | 60 | 60 | 60 | 60 | 60 | 60 |
| Citric Acid | 3 | 3 | 3 | 3 | 3 | 3 | 3 |
| Vitamin C | 0.2 | 0.2 | 0.2 | 0.2 | 0.2 | 0.2 | 0.2 |
| Sodium Citrate | 0.5 | 0.5 | 0.5 | 0.5 | 0.5 | 0.5 | 0.5 |
| Water up to | 1000 g | 1000 g | 1000 g | 1000 g | 1000 g | 1000 g | 1000 g |

TABLE 6
Beverage compositions H to N.

| | H | I | J | K | L | M | N |
|---|---|---|---|---|---|---|---|
| | Mass | Mass | Mass | Mass | Mass | Mass | Mass |
| Ingredients | (g) | (g) | (g) | (g) | (g) | (g) | (g) |
| Flavor | 1.19 | 1.25 | 1.19 | 1.43 | 1.43 | 1.25 | 1.10 |
| microemulsion | | | | | | | |
| Sugar | 60 | 60 | 60 | 60 | 60 | 60 | 60 |
| Citric Acid | 3 | 3 | 3 | 3 | 3 | 3 | 3 |
| Vitamin C | 0.2 | 0.2 | 0.2 | 0.2 | 0.2 | 0.2 | 0.2 |
| Sodium Citrate | 0.5 | 0.5 | 0.5 | 0.5 | 0.5 | 0.5 | 0.5 |
| Water up to | 1000 g | 1000 g | 1000 g | 1000 g | 1000 g | 1000 g | 1000 g |

TABLE 7
Comparative beverage compositions O and P.

| | O | P |
|---|---|---|
| Ingredients | Mass (g) | Mass (g) |
| Flavor nanoemulsion | 16.67 | 16.67 |
| Sugar | 60 | 60 |
| Citric Acid | 3 | 3 |
| Vitamin C | 0.2 | 0.2 |
| Sodium Citrate | 0.5 | 0.5 |
| Water up to | 1000 g | 1000 g |

Droplet sizes of all beverages were measured by a Zetasizer nano ZS (Malvern Instruments Limited, Worcs, UK). The turbidity of the beverages was measured by a portable turbidity meter (Hanna instruments, Woonsocket, R.I., HI93703) and was reported in Nephelometric Turbidity Unit (NTU).

Droplet sizes and turbidities as measured for the beverages are summarized in Tables 8-10 below:

TABLE 8
Droplet size and turbidity results for Beverages A to G.

| | A | B | C | D | E | F | G |
|---|---|---|---|---|---|---|---|
| Z-average* (nm) | 33.93 | 36.71 | 24.3 | 23.54 | 32.64 | 29.36 | 30.32 |
| Turbidity (NTU) | 4.46 | 5.89 | 3.73 | 3.06 | 6.03 | 4.05 | 5.72 |

*Z-average refers to the intensity weighted mean hydrodynamic diameter.

TABLE 9
Droplet size and turbidity results for Beverages H to N.

| | H | I | J | K | L | M | N |
|---|---|---|---|---|---|---|---|
| Z-average (nm) | 35.33 | 34.13 | 32.85 | 33.24 | 28.88 | 42.55 | 47.28 |
| Turbidity (NTU) | 5.55 | 4.13 | 6.04 | 7.37 | 3.94 | 5.88 | 9.84 |

TABLE 10
Droplet size and turbidity results
for Comparative Beverages O and P.

| | O | P |
|---|---|---|
| Z-average (nm) | 151.5 | 161.2 |
| Turbidity (NTU) | 224 | 300 |

Due to the low turbidity and droplet sizes, Beverages A to N were clear (translucent) in appearance. In comparison, Beverages O and P (comparative Beverages) had much higher droplet size and turbidity when applied at the same flavor dosage in the beverages and thus, Beverages O and P were not clear in appearance. Moreover, high energy input was required to prepare the comparative flavor nanoemulsions, and the oil loading was only 3%.

4. Physical stability of the flavor compositions

Physical stability of selected flavor compositions (Sample 2 and 12, Comparative Samples 1 and 2) was tested using LumiSizer (LUM GmbH, Germany). Thereby, stability of the samples is tested by accelerating the phase separation through centrifugation. The test was conducted at 20 to 25° C. with a centrifugation speed of 4000 rpm, the transmission of the samples was detected at 870 nm, a total of 900 profiles with an interval of 80 s was collected. Instability index calculated based on the change of transmission during centrifugation was determined, the lower the instability index, the higher the stability was.

TABLE 11
Instability index of the flavor compositions

| | | | Comparative | Comparative |
|---|---|---|---|---|
| | Sample 2 | Sample 12 | Sample 1 | Sample 2 |
| Instability index | 0.057 | 0.066 | 0.189 | 0.221 |

As shown in Table 11, the physical stability of the samples according to the present invention was significantly higher than that of the comparative samples.

5. Stability of the flavor compositions in acidic beverages during storage

Stability of selected flavor compositions based on different tonalities in acidic beverages (pH 3.2) during storage was tested at room temperature (20-25° C.) for one month. Beverages Q, R, S, T and U were prepared by diluting Samples 2, 3, 5, 6 and 12, respectively. The final flavor dosage was adjusted based on sensory evaluation. The composition of beverages and turbidity before and after storage for one month is given in Table 12.

TABLE 12
Beverages compositions and turbidity during storage.

| | Q | R | S | T | U |
|---|---|---|---|---|---|
| | Mass | Mass | Mass | Mass | Mass |
| Ingredients | (g) | (g) | (g) | (g) | (g) |
| Flavor | 0.088 | 0.11 | 0.11 | 0.11 | 0.11 |
| microemulsion | | | | | |
| Sugar | 60.0 | 60.0 | 60.0 | 60.0 | 60.0 |
| Citric Acid | 3.0 | 3.0 | 3.0 | 3.0 | 3.0 |
| Vitamin C | 0.2 | 0.2 | 0.2 | 0.2 | 0.2 |
| Sodium Citrate | 1.0 | 1.0 | 1.0 | 1.0 | 1.0 |
| Water up to | 1000 | 1000 | 1000 | 1000 | 1000 |
| Turbidity on | 0.90 | 1.06 | 1.57 | 2.07 | 1.25 |
| Day 0 (NTU) | | | | | |
| Turbidity on | 0.39 | 0.95 | 1.61 | 1.21 | 2.17 |
| Day 30 (NTU) | | | | | |

As shown in Table 12, the beverages showed good stability during storage at room temperature with only minor changes in the turbidity values.

6. Preparation of mouthwash using the flavor microemulsions

A sufficient amount of Sample 15 (corresponding to anyone of Samples 1 to 14 except that a menthol flavor has been added) is weighed and mixed in the mouthwash compositions according to Table 13 and Table 14, respectively, to add the equivalent of 0.24 wt. % flavor.

TABLE 13
Mouthwash formulation 1

| Ingredients | Amount (wt. %) |
|---|---|
| Propylene Glycol | 10% |
| Flavor | 0.240% |
| DI/Purified Water | up to 100% |
| Poloxamer 407 NF | 0.240% |
| Sodium Lauryl Sulfate | 0.040% |
| Sorbitol (70% solution) | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

TABLE 14
Mouthwash formulation 2

| Ingredients | Amount (wt. %) |
|---|---|
| Ethyl Alcohol 190 Proof | 15.0% |
| Flavor | 0.240% |
| DI/Purified Water | up to 100% |
| Poloxamer 407 NF | 0.240% |
| Sodium Lauryl Sulfate | 0.040% |
| Sorbitol (70% solution) | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

1. A composition comprising
a surfactant system comprising lysolecithin and lecithin, or lysolecithin and sugar ester,
a non-polar phase comprising a flavor oil,
a polar phase comprising at least one polar non-aqueous solvent; wherein the flavor oil is present in an amount of at least 10 wt. %, based on the total amount of the composition, and wherein lysolecithin is present in an amount of at least 10 wt. %, based on the total amount of the composition.

2. The composition according to claim 1, wherein the composition is an emulsion.

3. The composition according to claim 1, wherein the mass ratio of lysolecithin and lecithin is between 20:0 to 1:5.

4. The composition according to claim 1, wherein the mass ratio of lysolecithin and sugar ester is between 20:0 to 1:5.

5. The composition according to claim 1, wherein the polar phase further comprises water.

6. The composition according to claim 1, wherein the polar non-aqueous solvent is selected from the group consisting of glycerol, propylene glycol, benzylic alcohol, ethanol, propanol, isopropanol, 1,3-propanediol, butanol, butylene glycol, hexylene glycol, dipropylene glycol, ethoxydiglycol, triacetine, triethylcitrate, C1-6 linear/branched alkyl lactate (e.g. ethyl lactate), xylitol, sorbitol, mannitol, maltitol, inositol, allitol, altritol, dulcitol, galactitol, glucitol, hexitol, iditol, pentitol, ribitol, erythritol, and any mixtures thereof.

7. The composition according to claim 1, wherein the polar non-aqueous solvent is present in an amount of from 10 to 60 wt. %, based on the total amount of the composition.

8. The composition according to claim 1, wherein the flavor oil is present in an amount of at least 20 wt. %, based on the total amount of the composition.

9. The composition according to claim 8, wherein the flavor oil is present in the composition in an amount of from 20 to 50 wt. %, based on the total amount of the composition.

10. The composition according to claim 1, wherein lysolecithin is present in an amount of from 10 to 30 wt. %, based on the total amount of the composition.

11. A method for preparing a composition according to claim 1, comprising the steps of:
a. Mixing a non-polar phase comprising a flavor oil and a polar phase comprising at least one polar non-aqueous solvent in the presence of a surfactant system comprising lysolecithin and lecithin, or lysolecithin and sugar ester,
b. Stirring the mixture obtained after step a).

12. A method for preparing a flavored beverage or personal care product comprising the step of:
a. adding the composition according to claim 1 to a beverage or personal care product.

13. A beverage or personal care product comprising a composition according to claim 1.

14. The beverage according to claim 13, wherein the beverage is an alcoholic or non-alcoholic beverage.

15. The beverage according to claim 13, wherein the beverage shows a pH-value of from 2.5 to 7.

16. The beverage according to claim 13, wherein the beverage comprises a flavor oil in amounts of from 0.0001 to 0.1 wt. %, based on the total amount of the beverage.

17. The composition according to claim 2, wherein the composition is a microemulsion.

18. The composition according to claim 6, wherein the polar non-aqueous solvent is selected from the group consisting of glycerol, propylene glycol, ethyl lactate, ethanol, and sorbitol,

19. The composition according to claim 18, wherein the polar non-aqueous solvent is propylene glycol.

20. The beverage according to claim 15, wherein the beverage shows a pH-value of from 2.6 to 4.0.
